(19) United States
(12) Patent Application Publication
(10) Pub. No.: US 2021/0007659 A1
(43) Pub. Date: Jan. 14, 2021

(54) SYSTEM AND METHOD FOR SLEEP DISORDERS: SCREENING, TESTING AND MANAGEMENT

(71) Applicant: Teleplus Healthcare LLC, Summit, NJ (US)

(72) Inventors: Suzanne Shugg, Summit, NJ (US); Ree Cherng, Summit, NJ (US)

(21) Appl. No.: 16/915,697

(22) Filed: Jun. 29, 2020

Related U.S. Application Data
(60) Provisional application No. 62/868,438, filed on Jun. 28, 2019.

(51) Int. Cl. A61B 5/00; A61M 16/00; A61F 5/56; A61B 5/087; A61B 5/1455; A61B 6/00; G16H 50/30; G16H 50/20

(52) U.S. Cl. CPC A61B 5/4818; G16H 50/20; A61B 5/742; A61B 5/4815; A61B 5/7267; A61B 5/4848; A61B 5/0022; A61M 16/026; A61F 5/566; A61B 5/087; A61B 5/14551; A61B 5/4809; A61B 6/505; A61B 6/5211; G16H 50/30; A61B 5/7275

(57) ABSTRACT

The present invention provides a system and/or platform that can efficiently monitor/manage patients with sleep disorders. In one embodiment, the system and platform can train and/or certify (or help in training/certifying) service providers/professionals. In one embodiment, the system and platform is integrated with a software or an information system to manage data related to patients. In one embodiment, the system and platform utilizes home sleep test which is more convenient and acceptable. In one embodiment, the system/platform provides the information and knowledge related to a subject's conditions.

### CROSS-REFERENCE TO RELATED APPLICATIONS

This application claims the benefit of U.S. Provisional Application No. 62/868,438, filed Jun. 28, 2019. The entire contents and disclosures of the preceding application are incorporated by reference into this application.

Throughout this application, various publications are cited. The disclosures of these publications in their entireties are hereby incorporated by reference into this application to more fully describe the state of the art to which this invention pertains.

### FIELD OF THE INVENTION

This invention relates to a system and method for sleep disorders, in particular, sleep apnea.

### BACKGROUND OF THE INVENTION

Sleep apnea is a condition that makes a subject stop breathing for short periods while he/she is asleep. There are 2 types of sleep apnea. One is called “obstructive sleep apnea” (OSA) and the other is called “central sleep apnea” (CSA). In OSA, breathing during sleep is affected because of reduced or completely blocked airflow through airway. A completely blocked airway without airflow is called an obstructive apnea. Partial obstruction with diminished airflow is called a hypopnea. A person may have apnea and hypopnea during sleep. In CSA, breathing is stopped because the brain does not send the right signals to muscles to make the subject breathe. OSA currently affects approximately 40% of the of the Asian population. The majority of these people are unaware of such condition and do not take any preventive or therapeutic measures because of their lack of knowledge and resources. For example, Asia has no more than 200 trained Physicians in the field of sleep disorders by 2019.

The main symptoms of sleep apnea are loud snoring, tiredness, and daytime sleepiness. Other symptoms can include: restless sleep; waking up choking or gasping; morning headaches, dry mouth, or sore throat; waking up often to urinate; waking up feeling unrested or groggy; trouble thinking clearly or remembering things.

The causing factors, as defined as factors that increase the risk of obstructive sleep apnea (OSA), typically include:
Age—OSA occurs at all ages, but it is more common in middle and older age adults;
Gender—OSA is two times more common in men, especially in middle age;
Obesity—The more obese a person is, the more likely he or she is to have OSA;
Sedation from medication or alcohol—This interferes with the ability to awaken from sleep and can lengthen periods of apnea (no breathing), with potentially dangerous consequences; and
Abnormality of the airway.

It is well acknowledged that US is the world leader in implementing home sleep testing, diagnostics and/or interventions. Nonetheless, sleep disorders such as the sleep apnea are often poorly understood and neglected in practice due to factors including insufficient providers, insufficient software and physical equipment, public's lack of knowledge, and lack of effective intervention for a patient with various symptoms are impeding the development outside of US.

It is found that Asians have a higher propensity to have sleep problems due to their cranial facial structure and genetics. The prevalence of sleep disorders in the Asian population is 42%. However, less than 1% of patients are treated due to lack of information, education, and ready-to-use system to manage/monitor patients' information and resources.

Sleep disorders become a crucial issue in Taiwan. A survey in Taiwan by Tai et al. found that 46.6% of all participants had poor sleep quality and that 21.8% of individuals with poor sleep quality had used hypnotics to help them fall asleep in the past 4 weeks (1).

Tsou conducted an analysis in in elderly Taiwanese and found that there is a potential relevance between sleep issues and some behaviors such as smoking, alcohol consumption, physical inactivity and obesity. It further recognized the complexity of the relationship between sleep and other behaviors and suggested that “the directions of causality cannot be determined with the cross-sectional data used in this analysis” (2).

To the best knowledge of the inventor, there is no existing system or platform that can efficiently coordinate all parties of interest, and diagnose, treat, monitor and/or manage subjects with sleep disorders or subject to the risk therewith. In one embodiment, the subjects include these diagnosed and to be diagnosed, these treated, being treated and to be treated, and/or these recovered and under recovery. In one embodiment, the system and platform as disclosed and described in the present invention can train or help in training service providers/professionals. In one embodiment, the system and platform is capable of certifying providers/professionals that can provide relevant services. In one embodiment, the system and platform is integrated with a software or an information system to manage data related to patients. In one embodiment, the system and platform utilizes home sleep test which is more convenient and acceptable; while, polysomnography (PSG), as a standard multi-channel recording widely used is complex, uncomfortable, and time-consuming. (3) In one embodiment, the system/platform provides the information and knowledge related to a subject's conditions. In one embodiment, the system/platform of the present invention provides follow-ups in all stages including without limitation before diagnosis, before, during or after treatment, after partial or full recovery, and before and after re-admission.

Without timely diagnosis and treatment, people with sleep disorders may face higher risks, such as myocardial infarction, strokes, sudden cardiac death, atrial fibrillation, heart disease, dementia, Parkinson's disease, traffic accident.

U.S. Pat. No. US10,278,638B2 disclosed a sleep assist system to monitor and assist the user's sleep, and the system comprises: a bedside device adapted to be positioned near the user's bed, the bedside device optionally comprising a loudspeaker, a light source, a microphone, a light sensor, a temperature sensor, a control unit, an air quality sensor, a display unit, a user interface. However, such system is only to collect data as is and would be only capable of providing general information without particulars, and may not be able to detect any potential risks or provide any recommendation effectively relieving or treating the sleep disorder.

United States Patent Application Publication no. US20160151603A1 disclosed methods and systems for sleep management, and the system comprises a monitor such as a non-contact motion sensor to determine sleep information to be recorded, evaluated and/or displayed for the user. Further, the system may generate a sleep advice comprising content to promote good sleep habits and/or detect risky sleep conditions. However, such advice is generated by using limited information based on a fixed calculation, and would not be able to generate any recommendation effectively relieving or treating the sleep disorder.

In one aspect, the traditional approach requires an intensive amount of resources for calculation or exploration of relationships among a number of variables based on a huge volume of data being created and cumulated. The present invention provides an algorithm (or AI-based algorithm) by establishing predictive models in a relational database which can be ready to be retrieved for efficient use. In one embodiment, such AI-based algorithm establishes specific predictive models adaptable to the then-available data of the specific subject. In one embodiment, the establishment of the algorithm is performed via a selection module which selects from the predictive models that are adaptable to the specific subject. In one embodiment, the selection module can be either separate from the algorithm or part of the algorithm. In one embodiment, the selection module is implemented at the interface between the rational database and the databases storing data from data sources.

In one aspect, the data from different data sources is hard to integrate or translate into risks associated with sleep disorders. The present invention integrates data from data sources into a composite form and categorizes the risk levels by exploring the meaningful threshold values based on AI-algorithms. In one embodiment, the threshold values are adjusted by the AI-algorithms with data being cumulated. In one embodiment, the threshold values are created for a specific subject by the AI-algorithms with the then-available data.

In one aspect, the algorithm is dynamically evolving. After more and more data and predictive models are analyzed/established, the algorithm intelligently establishes or upgrades the way of integrating data and exploring predictive models. In one embodiment, the data can be integrated by fitting into one or more integration functions that are likely to generate meaningful composite (or overall) indicators. In one embodiment, the predictive models can be established by sequentially fitting into the exploration functions with the highest probability that can lead to a predictive model with target or satisfied accuracy.

Most patients have OSA because of a small upper airway. As the bones of the face and skull develop, some people develop a small lower face, a small mouth, and a tongue that seems too large for the mouth. These features are genetically determined, which explains why OSA tends to cluster in families. Obesity is another major factor. Tonsil enlargement can be an important cause, especially in children. In one aspect, traditional approaches for diagnosing sleep disorders do not consider race factor or genetic factor. In one embodiment, the present invention evaluates cranial facial structure's effect on sleep disorders. In one embodiment, a similarity function is established to determine the similarity of a subject's cranial facial structure with the one of an Asian. In one embodiment, the similarity function is a function of features of cranial facial structure. In one embodiment, the features comprise the size, shape, and configuration of the fourteen (14) facial bones including inferior nasal concha, lacrimal bones, mandible, maxilla, nasal bones, palatine bones, vomer, zygomatic bones. In one embodiment, the features of cranial facial structure are obtained by a facial X-ray. In one embodiment, the similarity function is a function of features of cranial facial structure and features of muscles and tissues surrounding throat. In one embodiment, the muscles and tissues include tongue, and tonsils. In one embodiment, an OSA is caused by a diminished or closed airway which is configurated by throat muscles during sleep. In one embodiment, the present invention correlates OSA with these features via such similarity function. In one embodiment, the present invention evaluates gene's effect on sleep disorders.

In one aspect, it is crucial to efficiently establish a correct and accurate predictive model for analysis or treatment of sleep disorders. The complexity of data such as high dimensionality cumbers such efficient establishment. In one embodiment, the present invention achieves so by using an algorithm that reduces the dimensionality according to their priorities or matching with the then-available data of a specific subject or a group of specific subjects.

It is well acknowledged that, in addition to existence and severity of sleep disorders, it is critical to identify the causes behind and an optimal option for treatment. In one embodiment, the present invention utilizes AI algorithms to identify the causes and the optimal option on a data-driven basis. In one embodiment, the algorithm provides a personalized treatment to a specific subject.

### SUMMARY OF THE INVENTION

In one embodiment, the present invention discloses a Home Sleep Apnea Test (HSAT) for screening or diagnosing OSA. In one embodiment, the present invention discloses a sleeping test device for the HSAT and a sleep therapy device for subjects in need. In one embodiment, the present invention discloses a method of generating analysis report based on cloud or AI-platform. In one embodiment, the present invention discloses a remote monitoring care for subjects in need.

In one embodiment, the present invention provides a Sleep Management Platform characterized for remote sleep testing, cloud or AI based analysis report, and/or remoted monitoring with a specific monitoring algorithm. In one embodiment, these Algorithms identify prioritized or optimal intervention and where and/or when the intervention is needed. In one embodiment, the platform comprises multiple sub-platforms:
1. a subsystem for onboarding patients with their medical information on chronic diseases, medications, allergies, and the provider information;
2. a subsystem for screening of sleep disorder that is specific to certain group of population, such as the Asian population;
3. a subsystem for coordinating HSAT and transmitting the result;
4. a subsystem for remote sleep monitoring;
5. a subsystem that stores management algorithms and remits records of a subject to a storage system accessible to management team and the user.

### Definitions and Abbreviations

The following terms shall be used to describe the present invention. In the absence of a specific definition set forth herein, the terms used to describe the present invention shall be given their common meanings as understood by those of ordinary skill in the art.

| Abbreviation | Definition |
|---|---|
| AI | Artificial Intelligence |
| AFIB | Atrial fibrillation |
| AHI | Apnea-Hypopnea Index |
| CHF | Congestive heart failure |
| COPD | Chronic obstructive pulmonary disease |
| CRSD | Circadian Rhythm Sleep Disorders |
| CSA | Central sleep apnea |
| EMR | Electronic Medical Record |
| ESS | Epworth Sleepiness Scale |
| HST (HSAT) | Home Sleep Test (Home Sleep Apnea Test) |
| IT | Information Technology |
| MD | Medical doctor |
| OHS | Obesity Hypoventilation Syndrome |
| OSA | Obstructive Sleep Apnea |
| PAP | Positive Air Pressure |
| PSG | Polysomnography |

### BRIEF DESCRIPTION OF THE FIGURES

FIG. 1 shows the concept of an education module that may be used in the present invention.

FIG. 2A shows a typical initial screening/assessment module of the present invention.

FIG. 2B shows a typical diagnostic loop of the present invention.

FIG. 2C shows a typical therapy (intervention) loop of the present invention.

FIG. 2D shows a typical monitoring loop of the present invention.

FIG. 3 depicts a typical workflow to calculate AHI for simple patients.

FIG. 4 shows a report of a subject.

### DETAILED DESCRIPTION OF THE INVENTION

In one embodiment, the system as disclosed in the present invention comprises four stages:
(1) Initial Screening;
(2) Diagnosis (diagnostic loop);
(3) Therapy intervention and titration; and
(4) Monitoring and/or management.

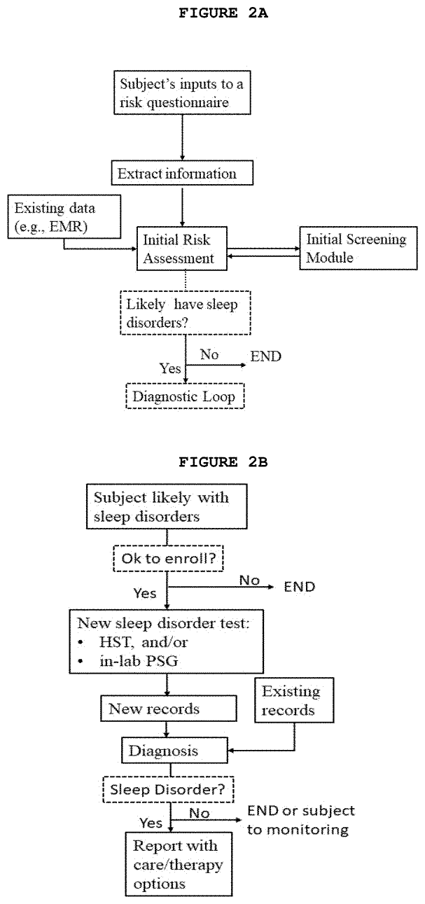

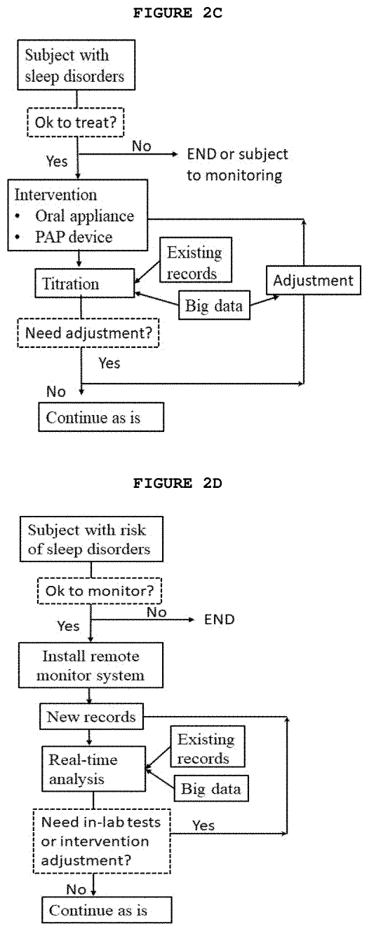

In one embodiment, the system comprises four modules for conducting 1) initial screening, 2) diagnosis, 3) therapy/intervention, and 4) monitoring and/or management by following the typical workflows in FIGS. 2A, 2B, 2C and 2D, respectively. In one embodiment, the system/platform of the present invention comprises one or more modules from the above four modules.

### Initial Screening/Assessment

In one embodiment, a typical initial screening is conducted as shown in FIG. 2A.

In one embodiment, a typical questionnaire is shown in Table 1. In one embodiment, the feedback can be collected remotely.

TABLE 1
Typical Questionnaire for Sleep Disorders

| | | |
|---|---|---|
| Item No. | Situation | Scale |

| | |
|---|---|
| 1. | Sitting and reading |
| 2. | Watching TV |
| 3. | Sitting inactive in a public place (e.g., a |
| | theatre or a meeting) |
| 4. | As a passenger in a car for an hour without a |
| | break |
| 5. | Lying down to rest in the afternoon when |
| | circumstances permit |
| 6. | Sitting and talking to someone |
| 7. | Sitting quietly after a lunch without alcohol |
| 8. | In a car, while stopped for a few minutes in |
| | traffic |
| 9. | When sitting and playing a game, such as |
| | Mahjong, poker, or chess |
| 10. | When working or studying in the late afternoon |
| | or in the early evening on the days without an |
| | after-lunch nap |
| | Total Scale: |

In one embodiment, the scale in Table 1 can be Epworth Sleepiness Scale as shown in Table 2.

TABLE 2
Epworth Sleepiness Scale

| Scale | Description |
|---|---|
| 0 | no chance of dozing |
| 1 | slight chance of dozing |
| 2 | moderate chance of dozing |
| 3 | high chance of dozing |

In one embodiment, 8 out of 10 items in Table 1 shall be selected and used for calculation. In one embodiment, as validated by the present invention, Asian people shall include items 1-7 and 10. Non-Asian population shall include items 1-8.

In one embodiment, when the total scale is higher than a threshold value, the subject likely has sleep disorders or faces certain risks associated with same. In one embodiment, Epworth Sleepiness Scale (ESS) is used. In one embodiment, if the total value of ESS is less than 10, it suggests that the subject likely has sufficient sleep. In one embodiment, if the total value of ESS is equal to or higher than 10 and no more than 15, it suggests that the subject likely suffers from excessive daytime sleepiness. In one embodiment, if the total value of ESS is equal to or higher than 16, it suggests that the subject likely in a dangerous stage of sleep disorders and shall seek professional help as soon as possible.

In one embodiment, in addition to the questionnaire, the present invention also weighs the existing MDR of such subject, such as weight, height, age, allergies, colds, and respiratory infections (including covid-19), nocturia, chronic pain, diabetes, obesity, neurological disorder, chronic obstructive pulmonary disease (COPD) and cardiovascular diseases.

If a person faces a certain risk of sleep apnea, the present invention may recommend a full or formal sleep study. In one embodiment, the sleep study is conducted at home, provided that the person has only low to medium risk of sleep apnea and does not have any of other conditions such as Congestive Heart Failure, Severe COPD, or Parkinson's disease. In one embodiment, the sleep study is preferably conducted at a facility such as sleep center, where the person is hooked up to different machines that monitor heart rate, breathing, and other body functions.

### Diagnostic Loop

Once the initial screening suggests a potential risk associated with sleep disorder for a subject, a typical diagnostic loop may be conducted as shown in FIG. 2B.

In one embodiment, the present invention classifies or evaluates risks associated with sleep disorders by a home sleep test (HST). The HST helps identify individuals that may need additional treatment or testing for sleep disorders due to other preexisting conditions or physical size. In one embodiment, HST is conducted either at home or in a facility. In one embodiment, HST is conducted remotely by the subject himself. In one embodiment, the present invention integrates the home test result with the questionnaire analysis (e.g., Table 1). The data generated by HST includes brain waves, eye movements, heart rate, breathing pattern, blood oxygen level, body position, chest and abdominal movement, limb movement, snoring and other noise during sleep.

In one embodiment, an analysis based on the integrated information is conducted to determine risk associated with sleep disorders. In one embodiment, such analysis is based on an algorithm that identifies the correlation between various symptoms and the risk associated with sleep apnea.

The home test device of the present invention is characterized by its connection to the monitoring system. In one embodiment, the monitoring system collects the data to be stored in one database which is further used for analysis. In one embodiment, once the test data is updated, the monitoring system will, in operation with the analysis module, generate feedback as necessary. In one embodiment, such feedback includes risk level associated with the sleep apnea, effectiveness induced by a treatment, and recommended option to minimize risk in the future.

In one embodiment, the present invention uses polysomnography (PSG) to evaluate as necessary. The data generated by PSG includes: brain waves, eye movements, heart rate, breathing pattern, blood oxygen level, body position, chest and abdominal movement, limb movement, snoring and other noise during sleep.

Apnea-Hypopnea Index (AHI) measures sleep apnea severity. The AHI is the sum of the number of apneas (pauses in breathing) plus the number of hypopneas (periods of shallow breathing) that occur, on average, each hour. From the AHI rating chart (Table 3), an index less that 5 is considered normal; an AHI value between 5 and 15 denotes a mild sleep apnea; an AHI value between 15 and 30 suggests a moderate condition; and an AHI value greater than 30 is considered severe.

TABLE 3
AHI severity rating chart

| AHI | Rating |
|---|---|
| <5 | Normal (no sleep apnea) |
| 5-15 | Mild sleep apnea |
| 15-30 | Moderate sleep apnea |
| >30 | Severe sleep apnea |

In one embodiment, the system of the present invention incorporates all factors into one or more indices.

In one embodiment, the present invention integrates all factors by following Equation 1:

Overall Indicator=Σᵢ₌₁^{m}fᵢ*Bᵢ*Fᵢ Equation 1,

wherein fᵢ refers to a positive coefficient in connection with the ith factor; Bᵢ refers to the Boolean coefficient in connection with the ith factor, when the ith factor does not exist or the test result shows no effect on sleep disorders, the value of Bᵢ is set as zero (0), otherwise, it is set as 1; and Fᵢ refers to a positive coefficient reflecting the severity of the ith factor.

In one embodiment, at the initial screening stage, the overall indicator is calculated exclusively in view of the questionnaire inputs, e.g., ESS total scale, since other tests or results may be not available. In one embodiment, when AHI result is available, the Overall Indicator may be calculated as

Overall Indicator=f₁*F₁+f₂*F₂,

wherein f₁ and f₂ refer to the coefficients in connection with ESS result and AHI result, respectively, and F₁ and F₂ refer to the ESS total scale and AHI value, respectively. In one embodiment, the corresponding threshold value of the Overall Indicator is calculated by following equation 1), i.e.,

Overall Indicator_{threshold}=f₁*F_{1,threshold}+f₂*F_{2,threshold}.

In one embodiment, when only ESS and AHI results are used for calculation, assuming f₁=0.2 and f₂=0.8, the threshold value indicating a severe condition is calculated as 0.2*16+0.8*30 =27.2.

In one embodiment, the coefficients and threshold values are determined and/or adjusted by an AI-based algorithm.

In one embodiment, an initial screening report is generated automatically and delivered to the subject and/or the professionals. In one embodiment, the result and the report are incorporated into a database.

In one embodiment, risks at different levels are marked by different colors. In one embodiment, the risks are classified into three categories, e.g., significant risk, elevated risk, and minimal risk. One is marked in red, the second one is marked in yellow, and the third one is marked in green. In one embodiment, an AHI value of at least 30 (red) is marked as red. In one embodiment, an AHI value between 15 and 30 is marked as yellow, while an AHI value of less than 15 is marked as green. In one embodiment, a subject categorized as red shall critically need PSG test in addition to an HST. In one embodiment, a subject categorized as green shall usually not need PSG test. In one embodiment, the necessity of a PSG test for subjects categorized as yellow is decided on a case-by-case basis by considering all relevant risk factors. In one embodiment, the risk factors comprise diseases comorbid conditions and the parameters describing the severity. The conditions include:
a. Neurological disorders: MS, Parkinson's disease, stroke etc.
b. Narcolepsy (need to see a specialist, we can have a list of the specialist),
c. CHF. A subject with a moderate to severe CHF condition may result in more than 12 visits to the doctor a year.
d. AFIB,
e. High blood pressure,
f. COPD/Asthma. A subject with a moderate to severe condition of COPD/Asthma will have to be on O2 at times or have to see doctor for more than once a year.
g. Obesity (BMI>35),
h. Preop evaluation for bariatric surgery,

In one embodiment, the test is conducted in view of a database which stores the medicine, brand names in connection with the diseases or conditions leading to a higher risk. In one embodiment, the medicine includes the cortisone, methylprednisolone (Medrol), prednisone and triamcinolone.

In one embodiment, the diagnosis is usually established in view of person's medical history, physical examination, and testing, including:
a) A complaint of snoring and ineffective sleep;
b) Neck size (greater than 17 inches in men or 16 inches in women) is associated with an increased risk of sleep apnea;
c) A small upper airway: difficulty seeing the throat because of a tongue that is large for the mouth;
d) High blood pressure, especially if it is resistant to treatment; and
e) If a bed partner has observed the patient during episodes of stopped breathing (apnea), choking, or gasping during sleep, there is a strong possibility of sleep apnea.

### Therapy/Intervention

In one embodiment, a typical therapy/intervention loop is conducted as shown in FIG. 2C.

There are a number of possible treatments including weight loss, sleep position control, and no use of alcohol. However, these possible treatments may not be effective though they can be performed feasibly.

The most effective treatment for sleep apnea is a device that keeps airway open during sleep. Treatment with this device is called “continuous positive airway pressure (CPAP) and the device is called CPAP device.

The second effective treatment is an oral appliance or mandibular advancement device placed in mouth during sleep. Such device can also keep the airway open.

Another treatment is surgery to open the airway. Surgical procedures reshape structures in the upper airways or surgically reposition bone or soft tissue. Uvulopalatopharyngoplasty (UPPP) removes the uvula and excessive tissue in the throat, including the tonsils, if present. Other procedures, such as maxillomandibular advancement (MMA), address both the upper and lower pharyngeal airway more globally.

UPPP alone has limited success rates (less than 50 percent) and people can relapse (when OSA symptoms return after surgery) (4). As a result, this surgery is only recommended in a minority of people and should be considered with caution. MMA may have a higher success rate, particularly in people with abnormal jaw (maxilla and mandible) anatomy, but it is the most complicated procedure. A newer surgical approach, nerve stimulation to protrude the tongue, has promising success rates in very selected people. Tracheostomy creates a permanent opening in the neck. It is reserved for people with severe disease in whom less drastic measures have failed or are inappropriate. Although it is always successful in eliminating obstructive sleep apnea, tracheostomy requires significant lifestyle changes and carries some serious risks (e.g., infection, bleeding, blockage). All surgical treatments require discussions about the goals of treatment, the expected outcomes, and potential complications.

In one embodiment, the intervention can be oral appliance, CPAP device, home therapy device, a surgery or any combination thereof. In one embodiment, the home therapy device is characterized by its connection to the monitoring system. In one embodiment, the surgery includes UPPP and MMA.

In one embodiment, the effects from such intervention are titrated during treatment. In one embodiment, upon analysis of the titration result, the system provides an adjustment to the intervention so as to ensure or maximize the therapeutic effects. In one embodiment, the adjustment is personalized in view of an analysis conducted with assistance of artificial intelligence (AI).

In one embodiment, the present invention implements different algorithms into the system to treat patients in different categories. For example, an intervention within 24 hours is required for patients categorized as severe conditions (red), an intervention within 72 hours is required for patients categorized as moderate conditions (yellow), while a monthly check is sufficient for people determined as normal or with minimal risk (green).

### Monitoring or Management

In one embodiment, a typical monitoring/management loop is conducted as shown in FIG. 2D.

In one embodiment, such monitoring and management module is configurated to monitor and manage patients remotely. In one embodiment, with more and more records, the analysis is conducted within a short time. In one embodiment, when an HST is connected to the subject, the analysis is conducted in a real-time manner.

In one embodiment, the monitoring system classifies patients into different categories. For example, patients in a mild sleep apnea and without any other health conditions can be categorized as simple patients. In one embodiment, the monitoring system relocates more resources to patients with severe conditions so as to provide feedback and/or alarm in a real-time or near real-time manner.

In one embodiment, the monitoring system collets the following information related to the therapy device:

(1) Type of Device,

(2) Type of Airflow Sensor,

(3) Type of Respiratory Sensor (due or single),

(4) O2 Saturation,

(5) Heart Rate,

(6) Optional parameters,

(7) Body positions,

(8) Sleep/Wake Time, and

(9) Snoring.

In one embodiment, the monitoring/management system of the present invention measures the following factors:
Compliance (As used herein, compliance refers to wearing a CPAP device at least one time within 48 hours);
Air leak;
Mask leak;
Machine Leak/Malfunction; and
Pulse Oxygen saturation.

In one embodiment, the monitoring/management system notifies the team/user in need with the following colors:
Compliance (more than 3 days red), 2 days yellow, otherwise green;
Air leak, (major red), minor yellow, none green;
Mask leak, (major red), minor yellow, none green;
Machine Leak/Malfunction: leak, (major red), minor yellow, none green;
Pulse Oxygen saturation (below 90% red) otherwise green; and
Elements can be added or removed from these algorithms.

In one embodiment, our present invention is integrated with other products such as RESMED products.

In one embodiment, the monitoring/management system provides Alerts categorized as Equipment-related and Clinic-related. In one embodiment, the equipment-related alerts cover everything that indicates equipment malfunction, or potentially prevents successful use of PAP equipment in delivery of a therapy or intervention. The clinic-related alerts cover those that indicate abnormal sleep patterns, or suggests the necessity of an intervention and/or adjustment.

In one embodiment, the home test device for HST also comprises a means, or is integrated into the monitoring system to verify the function and/or malfunction of the device or parts of the device. In one embodiment, the home therapy device for sleep disorders also comprises a means, or is integrated into the monitoring system to verify the function and/or malfunction of the device or parts of the device.

In one embodiment, the monitoring/management system comprises 1) ApneaLink Air device; 2) Oximeter, 3) Effort sensor, 4) Disposable oximeter finger sensor, 5) Belt, 6) Reusable oximeter finger sensor, 7) Oximeter belt clip, and 8) Nasal cannula.

In one embodiment, the present invention is established on artificial intelligence (AI)-based algorithms. In one embodiment, the present invention uses one or more databases to collect, acquire and/or extract data or information from: 1) initial screening, 2) existing and newly generated records; 3) other patient's historical data; 4) relationship among parameters that are used to describe severity and/or treatment effects in connection with symptoms related to sleep disorders.

In one embodiment, the AI-based algorithms identify the relationships among parameters and save these relationships in a relational database. In one embodiment, the AI-based algorithms are fixed or dynamically changed. In one embodiment, the AI-based algorithms continuously train the data and upgrade the relational database. In one embodiment, the AI-algorithms train the data and use the trained data to update the relational database.

With more and more parameters being considered, the calculation becomes intensive and requires more resources to keep the speed. In one embodiment, upon input of records of a subject, the AI-algorithms will automatically reduce the dimensionality by selecting the parameters in the subject's then-available measures/records so that a particular relationship can be established for such subject. In one embodiment, the particular relationship of the subject is saved in a sub-relational database subject to further change or update in view of new records.

### Workflow to Calculate AHI for Simple Patients

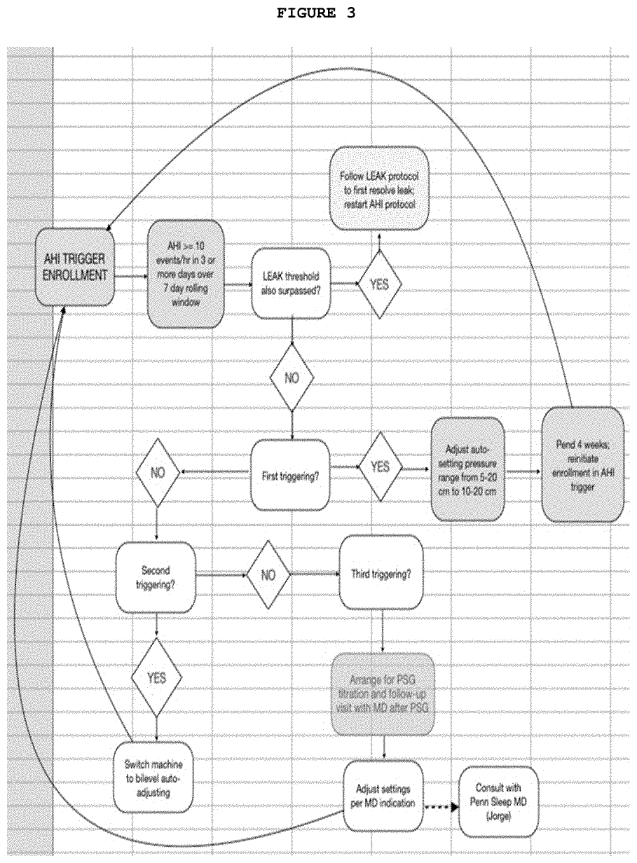

A typical workflow to calculate AHI is shown in FIG. 3.

The present invention provides a system for identifying and dynamically monitoring sleep disorders in a subject. the system comprises (1) a processing engine configured to interface with a plurality of data sources, wherein at least one data source comprises a database related to the subject, at least one data source comprises a database related to a plurality of subjects, at least one data source comprises a relational database comprising a set of predictive models describing the relationships between causing factors and risks associated with sleep disorders; (2) an analysis engine configured to (a)establish or update said set of predictive models in said relational database by interfacing with one or more databases of said plurality of data sources, and (b) dynamically analyze, in response to an input from a user or an update of one or more of said data sources, the risk of sleep disorders in said subject; (3) a reporting engine configured to generate a sleep disorder reporting interface and update the database related to the subject; (4) one or more computer-readable storage devices configured to store a plurality of computer executable instructions; and (5)one or more computer processors in communication with the one or more computer-readable storage devices and configured to execute the plurality of computer executable instructions in order to cause the system to (a) collect and aggregate data of the subject from one or more of the plurality of data sources, (b) establish, by the analysis engine, one or more specific predictive models that are adaptable to the aggregated data of the subject, wherein said one or more specific predictive models are used to identify the existence, severity and cause of sleep disorders in the subject, and (c) generate, by the reporting engine, a sleep disorder reporting interface comprising a visual representation of the existence, severity and cause of sleep disorders in said subject.

In one embodiment, the one or more predictive models in said relational database are established by said analysis engine with the assistance of an artificial intelligence algorithm which (a) receives, via interfacing with said plurality of data sources, a training dataset including: (1) a plurality of causing factors, (2) a plurality of impact factors that describe the effects of said causing factors on sleep disorders, and (3) one or more threshold values of one or more indices that indicate the existence, severity and cause of sleep disorders in a subject; and (b) trains, based on at least a subset of the training dataset, one or more predictive models configured to predict the existence, severity and cause of sleep disorders in a subject with one or more causing factors.

In one embodiment, the one or more predictive models in said relational database are established by said analysis engine with the assistance of an artificial intelligence algorithm which (a) receives a training dataset including: (1) a plurality of causing factors, (2) a plurality of impact factors that describe the effects of said causing factors on sleep disorders, (3) a plurality of treatment options, (4) a plurality of treatment factors that scale the therapeutic effects of said treatment options, and (5)one or more threshold values of one or more indices that indicate the existence, severity and cause of sleep disorders in the subject and indicate treatment effects from one or more treatment options; and (b) trains, based on at least a subset of the training dataset, said one or more predictive models configured to predict the existence, severity and cause of sleep disorders in the subject with one or more causing factors and predict treatment effects from one or more treatment options.

In one embodiment, the artificial intelligence algorithm further establishes, based on the aggregated data of the subject, one or more specific additional predictive models adaptable for the subject.

In one embodiment, the plurality of treatment options are selected from the group consisting of weight loss, sleep position control, no intake of alcohol, CPAP, an oral appliance, and surgery.

In one embodiment, one or more of said predictive models describe the relationships between multiple causing factors and their effects by using the Overall Indicator:

$$Overall Indicator = {\sum }_{i = 1}^{m} {f}_{i} * {B}_{i} * {F}_{i}$$

wherein fᵢ is a positive coefficient of the ith causing factor; and Bᵢ is a Boolean coefficient of the ith causing factor, wherein when the ith causing factor does not exist or the test result shows no effect on sleep disorders, the value of Bᵢ is set as zero (0), and Bᵢ is set as 1 if the ith causing factor exists and the test result shows an effect on sleep disorders; and Fᵢ is a positive scale or value reflecting the importance of the ith factor.

In one embodiment, the data in the database related to a plurality of subjects comprise data of general public with or without sleep disorders.

In one embodiment, the one or more specific predictive models are selected from said set of predictive models, or established by a dimensionality-reduced algorithm, wherein variables in the aggregated data are selected or prioritized to ensure efficiency and accuracy of the analysis.

In one embodiment, the sleep disorder reporting interface further comprises a recommendation for one or more tests or treatments.

In one embodiment, the data in at least one of said plurality of data sources comprise features related to cranial facial structure and genetics, and at least one of said data sources is connected via a cable or a network to a test device.

In one embodiment, one or more of the predictive models takes into consideration the cranial facial structure and genetics of an Asian subject as a causing factor that may lead to high risk of sleep disorders in Asian subjects.

In one embodiment, the system further comprises an output-on-demand interface secured for designated professional, wherein said output-on-demand interface, upon an input from said designated professional, displays relevant information or record, directly or via another output interface linked to said relevant information or record.

In one embodiment, the present invention provides a platform for identifying, monitoring and treating sleep disorders in a subject. The platform comprises the above-mentioned system for identifying and dynamically monitoring sleep disorders in a subject and a therapy device coupled to said system.

In one embodiment, the therapy device is selected from the group consisting of CPAP device and an oral appliance, said therapy device subject to further adjustment in view of recommendation according to said one or more specific predictive models.

In one embodiment, the present invention provides a system for monitoring sleep-related data and managing subjects with sleep disorders. The system comprises (a) a server that, in operation, facilitates interaction with subjects having sleeping disorder to contribute subject-specific data; (b) a database maintained by an administrative entity that, in operation, stores and aggregates the subject-specific data transmitted by each of said subjects; (c) a processing engine maintained by the administrative entity that, in operation, processes subject-specific data received from the subjects via one or more interfaces to establish subject-specific accounts based on the subject-specific data, and attributes a subject-specific risk value to the subject-specific accounts based upon respective subject-specific data; (d) a set of devices for monitoring sleep-related data of and provide an intervention to each of said subjects for a test period, wherein said set of devices contributes sleep-related data to said processing engine via said one or more interfaces; and (e) a template stored in said database comprising a set of anticipated events, wherein the processing engine analyzes the sleep-related data of each subject to determine at least one anticipated event before automatically and without human intervention, conduct one or more of the following: (i) sending follow-up instructions to each set of devices based upon said template to adjust said intervention; (ii) determining frequency of each of said anticipated events within said test period and reevaluates said subject-specific risk value; and (iii) sending follow-up communications comprising a custom report adapted to facilitate each subject to consult a medical professional.

In one embodiment, said subject-specific data is provided to the server via blockchain and comprises a score from the Epworth sleepiness scale and/or one or more risk factors selected from the group consisting of neurological disorder, narcolepsy, CHF, AFIB, high blood pressure COPD/Asthma, and obesity.

In one embodiment, the set of devices comprises polysomnography device, airflow sensor, respiratory sensor, continuous positive airway pressure machine, oximeter, and nasal cannula.

In one embodiment, the subject-specific risk value is based on Apnea-Hypopnea Index.

In one embodiment, the set of anticipated events comprises sleep/wake time, body positions, snoring, or apnea.

In one embodiment, the template is based upon analysis of the subject-specific data transmitted by all of said subjects or the subject-specific data transmitted by subjects having a similar condition or symptoms.

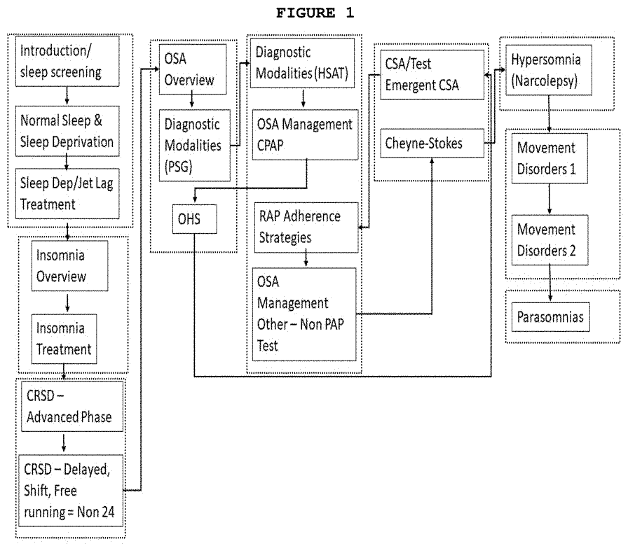

In one embodiment, professionals (including physicians and nurses) shall be certified prior to providing services. A certification is required for professionals or designated staff to screen, test and/or manage sleep disorders using the platform provided by the present invention. In one embodiment, the present invention relies an education module for certification of the professionals and staff. In one embodiment, the education module is representatively shown in FIG. 1. As used herein, the education module provides patient with the information and knowledge related to the subject's conditions or upon request from a user. In one embodiment, the education module is segmented into 9 sections as shown by the boxes in dot lines. In one embodiment, the education module is an online platform, e.g., https://www.tph-academy.com/. In one embodiment, the education module is integrated into an online platform that provides relevant information and/or knowledge in response to an inquiry. In one embodiment, the inquiry is from a user or the system.

In one embodiment, a program for certification by the education module covers materials similar to those offered by the UPENN MD certification upon completion of over 20 online modules with advanced analytics and testing (5). In one embodiment, the program for certification by the education module covers materials similar to the Education and Training of Sleep Medicine as offered by the Penn Medicine (6).

### EXAMPLES

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### Example 1

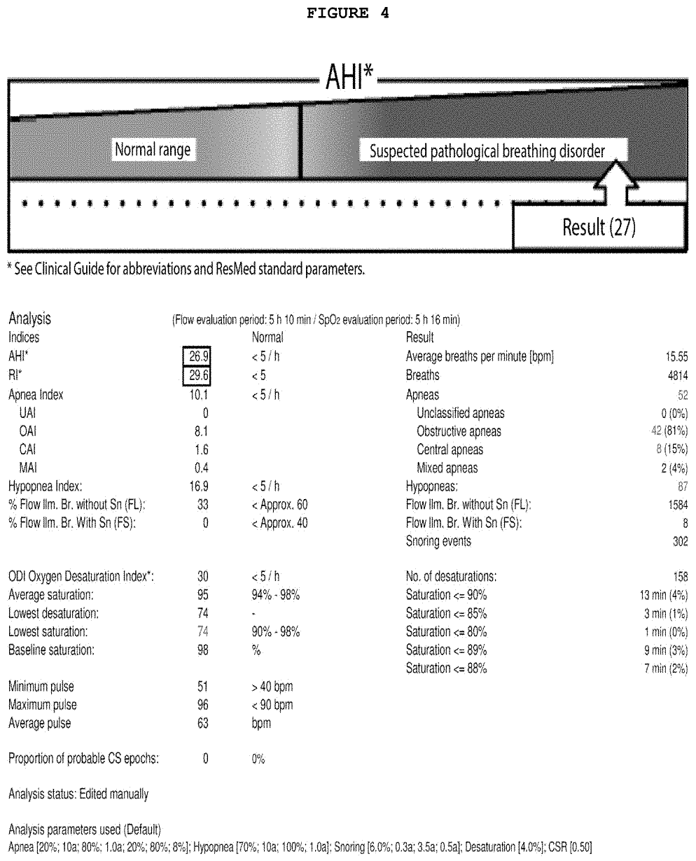

In this example, based on the report as shown in FIG. 4 and the AI algorithm, the present invention may identify the causes and recommend an optimal treatment for sleep disorders.

### REFERENCES

1. Tai S Y, Wang W F, Yang Y H. Current status of sleep quality in Taiwan: a nationwide walk-in survey. Annals of general psychiatry. 2015 Dec. 1; 14(1):36.

2. Tsou M T. Association between sleep dura outcome in elderly Taiwanese. International Journal of Gerontology. 2011 Dec. 1; 5(4):200-5.

3. Huang C S, Lin C L, Ko L W, Liu S Y, Su T P, Lin C T. Knowledge-based identification of sleep stages based on two forehead electroencephalogram channels. Frontiers in neuroscience. 2014 Sep. 4; 8:263.

4. Sean M Caples, James A Rowley, Jeffrey R Prinsell, John F Pallanch, Mohamed B Elamin, Sheri G Katz, John D Harwick. Surgical Modifications of the Upper Airway for Obstructive Sleep Apnea in Adults: A Systematic Review and Meta-Analysis. Sleep. 2010 October; 33(10):1396-407.

5. Continuing Medical Education by Center for Sleep and Circadian Neurobiology at Perelman School of Medicine, University of Pennsylvania (website: https://www.med.upenn.edu/sleepctr/cme.html).

6. Education and Training of Sleep Medicine by Department of Medicine at the Penn Medicine (website: https://www.pennmedicine.org/departments-and-centers/department-of-medicine/divisions/sleep-medicine/eduation-and-training).

What is claimed is:

1. A system for identifying and dynamically monitoring sleep disorders in a subject, the system comprising:
1) a processing engine configured to interface with a plurality of data sources, wherein at least one data source comprises a database related to the subject, at least one data source comprises a database related to a plurality of subjects, at least one data source comprises a relational database comprising a set of predictive models describing the relationships between causing factors and risks associated with sleep disorders;
2) an analysis engine configured to:
a) establish or update said set of predictive models in said relational database by interfacing with one or more databases of said plurality of data sources; and
b) dynamically analyze, in response to an input from a user or an update of one or more of said data sources, the risk of sleep disorders in said subject;
3) a reporting engine configured to generate a sleep disorder reporting interface and update the database related to the subject;
4) one or more computer-readable storage devices configured to store a plurality of computer executable instructions; and
5) one or more computer processors in communication with the one or more computer-readable storage devices and configured to execute the plurality of computer executable instructions in order to cause the system to:
a) collect and aggregate data of the subject from one or more of the plurality of data sources;
b) establish, by the analysis engine, one or more specific predictive models that are adaptable to the aggregated data of the subject, wherein said one or more specific predictive models are used to identify the existence, severity and cause of sleep disorders in the subject; and
c) generate, by the reporting engine, a sleep disorder reporting interface comprising a visual representation of the existence, severity and cause of sleep disorders in said subject.

2. The system of claim 1, wherein said one or more predictive models in said relational database are established by said analysis engine with the assistance of an artificial intelligence algorithm which:
a) receives, via interfacing with said plurality of data sources, a training dataset including:
(1) a plurality of causing factors,
(2) a plurality of impact factors that describe the effects of said causing factors on sleep disorders, and
(3) one or more threshold values of one or more indices that indicate the existence, severity and cause of sleep disorders in a subject; and
b) trains, based on at least a subset of the training dataset, one or more predictive models configured to predict the existence, severity and cause of sleep disorders in a subject with one or more causing factors.

3. The system of claim 1, wherein said one or more predictive models in said relational database are established by said analysis engine with the assistance of an artificial intelligence algorithm which:
a) receives a training dataset including:
(1) a plurality of causing factors,
(2) a plurality of impact factors that describe the effects of said causing factors on sleep disorders,
(3) a plurality of treatment options,
(4) a plurality of treatment factors that scale the therapeutic effects of said treatment options, and
(5) one or more threshold values of one or more indices that indicate the existence, severity and cause of sleep disorders in the subject and indicate treatment effects from one or more treatment options, and
b) trains, based on at least a subset of the training dataset, said one or more predictive models configured to predict the existence, severity and cause of sleep disorders in the subject with one or more causing factors and predict treatment effects from one or more treatment options.

4. The system of claim 3, wherein said artificial intelligence algorithm further establishes, based on the aggregated data of the subject, one or more specific additional predictive models adaptable for the subject.

5. The system of claim 3, wherein said plurality of treatment options are selected from the group consisting of weight loss, sleep position control, no intake of alcohol, CPAP, an oral appliance, and surgery.

6. The system of claim 3, wherein one or more of said predictive models describe the relationships between multiple causing factors and their effects by using the Overall Indicator:
$$Overall Indicator = {\sum }_{i = 1}^{m} {f}_{i} * {B}_{i} * {F}_{i}$$
wherein fᵢ is a positive coefficient of the ith causing factor; and Bᵢ is a Boolean coefficient of the ith causing factor, wherein when the ith causing factor does not exist or the test result shows no effect on sleep disorders, the value of Bᵢ is set as zero (0), and Bᵢ is set as 1 if the ith causing factor exists and the test result shows an effect on sleep disorders; and Fᵢ is a positive scale or value reflecting the importance of the ith factor.

7. The system of claim 1, wherein the data in the database related to a plurality of subjects comprise data of general public with or without sleep disorders.

8. The system of claim 1, wherein said one or more specific predictive models are selected from said set of predictive models, or established by a dimensionality-reduced algorithm, wherein variables in the aggregated data are selected or prioritized to ensure efficiency and accuracy of the analysis.

9. The system of claim 1, wherein the sleep disorder reporting interface further comprises a recommendation for one or more tests or treatments.

10. The system of claim 1, wherein the data in at least one of said plurality of data sources comprise features related to cranial facial structure and genetics, and at least one of said plurality of data sources is connected via a cable or a network to a test device.

11. The system of claim 1, wherein one or more of the predictive models takes into consideration the cranial facial structure and genetics of an Asian subject as a causing factor that may lead to high risk of sleep disorders in Asian subjects.

12. The system of claim 1, wherein said system further comprises an output-on-demand interface secured for designated professional, wherein said output-on-demand interface, upon an input from said designated professional, displays relevant information or record, directly or via another output interface linked to said relevant information or record.

13. A platform for identifying, monitoring and treating sleep disorders in a subject, the platform comprising:
a) the system of claim 3; and
b) a therapy device coupled to said system.

14. The platform of claim 13, wherein said therapy device is selected from the group consisting of CPAP device and an oral appliance, said therapy device subject to further adjustment in view of recommendation according to said one or more specific predictive models.

15. A system for monitoring sleep-related data and managing subjects with sleep disorders comprising:
a. a server that, in operation, facilitates interaction with subjects having sleeping disorder to contribute subject-specific data;
b. a database maintained by an administrative entity that, in operation, stores and aggregates the subject-specific data transmitted by each of said subjects; and
c. a processing engine maintained by the administrative entity that, in operation, processes subject-specific data received from the subjects via one or more interfaces to establish subject-specific accounts based on the subject-specific data, and attributes a subject-specific risk value to the subject-specific accounts based upon respective subject-specific data;
d. a set of devices for monitoring sleep-related data of and provide an intervention to each of said subjects for a test period, wherein said set of devices contributes sleep-related data to said processing engine via said one or more interfaces;
e. a template stored in said database comprising a set of anticipated events;
wherein the processing engine analyzes the sleep-related data of each subject to determine at least one anticipated event before automatically and without human intervention, conduct one or more of the following:
i. sending follow-up instructions to each set of devices based upon said template to adjust said intervention;
ii. determining frequency of each of said anticipated events within said test period and reevaluates said subject-specific risk value; and
iii. sending follow-up communications comprising a custom report adapted to facilitate each subject to consult a medical professional.

16. The system of claim 15, wherein said subject-specific data is provided to the server via blockchain and comprises a score from the Epworth sleepiness scale and/or one or more risk factors selected from the group consisting of neurological disorder, narcolepsy, CHF, AFIB, high blood pressure COPD/Asthma, and obesity.

17. The system of claim 15, wherein the set of devices comprises polysomnography device, airflow sensor, respiratory sensor, continuous positive airway pressure machine, oximeter, and nasal cannula.

18. The system of claim 15, wherein the subject-specific risk value is based on Apnea-Hypopnea Index.

19. The system of claim 15, wherein said set of anticipated events comprises sleep/wake time, body positions, snoring, or apnea.

20. The system of claim 15, wherein the template is based upon analysis of the subject-specific data transmitted by all of said subjects or the subject-specific data transmitted by subjects having a similar condition or symptoms.
